(19) United States
(12) Patent Application Publication
(10) Pub. No.: US 2018/0344382 A1
(43) Pub. Date: Dec. 6, 2018

(54) SURGICAL VAPORIZATION ELECTRODE

(71) Applicant: OLYMPUS WINTER & IBE GMBH, Hamburg (DE)

(72) Inventors: Christian BROCKMANN, Hollenstedt (DE); Thomas FREITAG, Hamburg (DE); Christoph KNOPF, Lübeck (DE)

(73) Assignee: OLYMPUS WINTER & IBE GMBH, Hamburg (DE)

(21) Appl. No.: 15/778,989

(22) PCT Filed: Dec. 8, 2016
(86) PCT No.: PCT/DE2016/000432

§ 371 Date: May 24, 2018

(30) Foreign Application Priority Data
Dec. 11, 2015 (DE) 10 2015 016 060.5

(51) Int. Cl. A61B 18/14; A61B 18/12; A61B 18/04

(52) U.S. Cl. CPC A61B 18/14; A61B 2018/00625; A61B 18/042; A61B 18/1206

(57) ABSTRACT

The electrode head includes two working surfaces in accordance with an actual bipolar electrode. These may be manufactured lithographically, exhibiting even more complicated outlines. Working surfaces are depicted, which are structured as annulus sector-shaped, concentrically arranged areas, when projected in a plane. Moreover, a further area is situated centrally, which is disc-shaped in a planar projection. Plasma is ignited alternately at both poles. If the individual concentric zones are situated close enough with respect to each other a continuous plasma layer will result.

### TECHNICAL AREA

The invention relates to a surgical vaporization electrode.

### STATE OF THE ART

Electric surgical resection instruments are known from the prior art, wherein during resection, radiofrequency (RF) alternating current is passed through the body part to be treated in order to remove or cut selectively the respective local tissue. In particular, this kind of resection instrument is used e.g. to remove adenomatous tissue by vaporization. For this purpose, an RF voltage is applied to an electrode, the RF voltage being generated by means of suitable RF generators and connected to the working part of the electrode via appropriate supply lines, wherein such electrodes, depending on the type, may be operated in a bipolar or a monopolar operating mode.

Most frequently, the monopolar operating mode is used, wherein one pole of the RF voltage generator is connected to the patient as a passive electrode, covering an area as large as possible, and wherein the surgical instrument (active electrode) is forming the other pole. The current flows via a path of least resistance from the active electrode to the passive electrode, such that current density is highest in the immediate vicinity of the active electrode. Here, the thermal effect is most pronounced in consequence, but also the surrounding tissue is heated due to current flow.

In the bipolar operating mode, the current flows through a small part of the body only, in contrast to the monopolar mode. The localized current density at the bipolar electrode causes rapid heating of the tissue surrounding the electrode head, resulting in vaporization of interstitial fluid or of the irrigation solution, which surrounds the tissue (saline).

A thin gas layer (vapor cushion) forms around the tip of the electrode, which can be ionized at sufficiently high voltage to generate stable plasma (plasma ignition). The energy of the plasma transfers to the cells of the tissue to be resected and leads to its localized vaporization. Using plasma vaporization, tissue may be separated and removed, respectively, in a more gentle and efficient way compared to conventional vaporization (e.g. using monopolar vaporization or laser evaporation), since plasma vaporization requires only minimal contact between the electrode and the tissue and does not necessitate high temperatures (“cold vaporization”).

In fact, conventional electrodes operate in a quasi-bipolar mode with an active (RF-voltage supplied) electrode and a return electrode. Therein, the return electrode is significantly larger than the active electrode, such that the plasma ignites only at the active electrode. For some conventional electrodes, the fork tubes holding the electrode head serve as return electrode, while the current is returned to the generator via the transporter. As is known to those skilled in the art, a transporter refers to an accessory instrument enabling the controlled movement of the electrode. Other conventional bipolar electrodes comprise a return electrode, which is insulated with respect to the electrode shaft, for returning current through the electrode. These electrodes also operate in a quasi-bipolar mode, since only one pole is configured as an active, RF-voltage supplied electrode.

Basically, the further removed from the electrode, the higher are the currents flowing through the patient. For quasi-bipolar electrodes, only a small portion of the current flows through the patient back into the electrode shaft rather than into the fork tubes via the saline solution.

The size of the active surface (working surface) constitutes another disadvantage of conventional vaporization electrodes. The larger the surface area at which the plasma is ignited, the more heat is conveyed to the surrounding saline solution. In order to obtain a higher vaporization rate, it is not sufficient to merely enlarge the active surface. A larger active surface also worsens ignitability.

### DETAILED DESCRIPTION OF THE INVENTION

Starting from the aforementioned electrodes of the state-of-the-art, it is the object of the present invention to provide for a device which does either not exhibit the disadvantages mentioned above or at least to a lesser extent.

The invention relates to a surgical vaporization electrode comprising an electrode head with at least two electrically conductive working surfaces, arranged as to be electrically isolated from each other. To this purpose, the working surfaces corresponding to the poles of the electrode may be applied in layers to an electrically non-conductive base member, e.g. by means of etching, sputtering, deposit welding, soldering, electrochemical coating or other coating techniques. Alternatively, the electrode head is composed of a plurality of sub-components, each of which is conductive, and isolated from the others, wherein each sub-component comprises one of the working surfaces. Regarding the working surfaces, materials already known from the state-of-the-art are considered here in particular.

According to a preferred embodiment, each of the working surfaces has at least one surface portion being substantially annulus-shaped, annulus sector-shaped, elliptical annulus-shaped or elliptical annulus sector-shaped, when projected in a plane, and the surface portions are arranged concentrically or approximately concentrically in relation to each other, when projected in a plane. In particular, approximately concentric is construed such that the circle-centers or the ellipse-centers of the annuli or annulus sectors, respectively, do not deviate from each other by more than 20%, preferably by not more than 10%, of their respective circle or largest ellipse diameter. With respect to substantially annulus sector-shaped or elliptical annulus sector-shaped surface portions it may be disregarded in general, as to how the surface edge extending from the respective outer annulus or elliptical annulus sector, defining the annulus sector, to the respective inner annulus sector or elliptical annulus sector, defining the annulus sector, is arranged specifically. Advantageously, other, elongated, curved surface portions at least partially surrounding each other may be provided, in particular crescent-shaped or involute curved surface portions.

According to an advantageous embodiment of the invention, a surgical instrument is provided, which comprises an RF surgical generator according to the invention, wherein the RF generator is configured and connected to the surgical vaporization electrode as to allow for activation and deactivation of the working surfaces separately from each other. Therein, activation and deactivation is construed as being supplied with high-frequency AC voltage or being separated from high-frequency AC voltage. This may be accomplished, in particular, in that each working surface is supplied with a separate electric supply line, which is connected to a high-frequency AC voltage source via a switch or electronic switching module, such as a relay, known per se from electrical engineering. Alternatively, each working surface e.g. may be connected via a corresponding supply line with its own high-frequency AC voltage source, which may be switched on and off.

Preferably, the surgical instrument comprises an electronic control for activating and deactivating the working surfaces. In principle, such electronic control devices known per se from the prior art are suitable, which are capable of controlling electronic switching modules associated with the working surfaces or of controlling high-frequency AC voltage sources associated with the working surfaces, respectively.

According to a preferred embodiment, the surgical instrument further comprises movement detection means for detecting a relative movement of the electrode head with respect to a reference system, wherein the electronic control is adapted to activate and/or deactivate at least one of the working surfaces depending upon the relative movement of the electrode head. To this end, e.g. the transporter may serve as reference system. For example, the relative movement of the electrode head with respect to the transporter may be determined indirectly as a relative movement of the electrode shaft with respect to the transporter. A plurality of movement detection sensors known per se from the prior art are suitable for this purpose, for example capacitive, magnetic or optical sensors. It is particularly preferred to arrange the sensors within reusable parts, e.g. in the transporter, and not in the electrodes, which are disposable instruments. In order to detect the movement of the electrode tip towards the optics, an indirect measurement of the carriage of the transporter (Teflon body) in relation to the rigid base member of the transporter (optical disk, cone, reinforcing tube, etc.) may be conducted advantageously.

Preferably, the electronic control is configured to activate at least one working surface leading with respect to the direction of movement of the electrode head and to deactivate at least one working surface trailing with respect to the direction of movement of the electrode head.

According to a further preferred embodiment, the surgical instrument comprises means for measuring impedance, wherein the electronic control is configured to activate and/or deactivate at least one of the working surfaces depending upon the impedance measurements. By means of sensors known per se known from the prior art, it is therefore determined by means of said impedance measurements, which of the working surfaces exhibits tissue contact. Working surfaces which are not contacting tissue may be disabled.

According to a further preferred embodiment, the surgical instrument is configured such that for plasma ignition, a predetermined working surface is activated prior to the activation of one or more of the remaining working surfaces.

The invention will be explained in more detail by way of examples with reference to the accompanying schematic figures. The figures are not drawn to scale; in particular, for reasons of clarity the respective ratios of the individual dimensions do not necessarily correspond to the dimensional ratios in actual technical implementations.

Several preferred embodiments are described, to which the invention is not limited, however. In principle, any variant of the present invention described or implied, respectively, in the context of the present application may be particularly advantageous, depending on economic, technical and, optionally, medical circumstances in any particular case. Unless stated otherwise, or as far as technically feasible, respectively, individual features of the embodiments described may be interchanged or combined with each other as well as with features known per se from the state-of-the-art.

### BRIEF DESCRIPTION OF THE FIGURES

FIG. 1a shows a cross-sectional view of an embodiment of the electrode head of a surgical vaporization electrode according to the invention.

FIG. 1b depicts the lower surface of the electrode head of the vaporization electrode from FIG. 1a in a plan view (from below), the sectional plane being indicated by line A-A′.

In FIG. 2 shows a cross-sectional view, similar to that in FIG. 1a, of a further embodiment of the electrode head of a surgical vaporization electrode according to the invention is shown, the plan view of which resembles FIG. 1b.

FIG. 3 depicts a cross-sectional view, similar to those in FIGS. 1a and 2, of a further exemplary embodiment of the electrode head of a surgical vaporization electrode according to the invention, the plan view of which, again, resembles FIG. 1b.

In FIG. 4a shows an embodiment of the electrode head of another surgical vaporization electrode according to the invention is depicted in a cross-sectional view, and, furthermore, the connection of the working surfaces to further components of a surgical instrument according to the invention.

FIG. 4b shows the lower surface of the electrode head of the vaporization electrode of FIG. 4a in a plan view (from below), the sectional plane being indicated by the line A-A′.

FIG. 5 shows the lower surface of the electrode head of a further vaporization electrode according to the invention in a top view (from below).

### PREFERRED EMBODIMENT OF THE INVENTION

Corresponding elements are denoted by the same respective reference numerals in the figures.

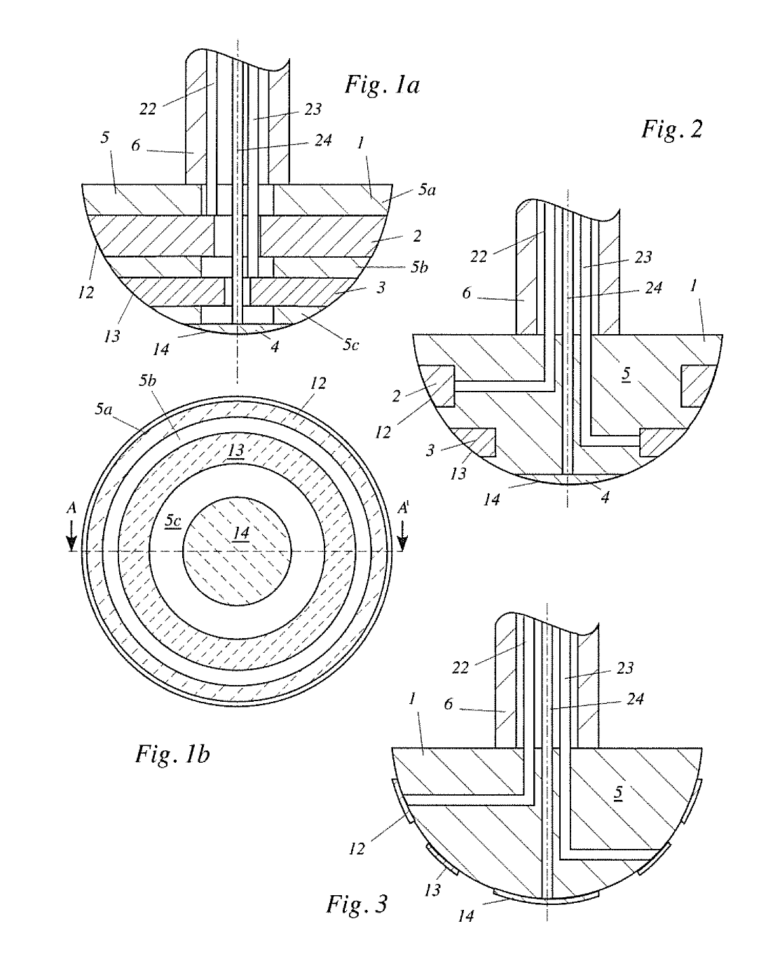

FIG. 1a shows an embodiment of the electrode head 1 of a surgical vaporization electrode according to the invention. In FIG. 1b, in the plan view of the lower surface the sectional plane A-A′ is indicated as a dashed line. The electrode head 1 consists of three metallic electrode bodies 2, 3, 4 and an insulator body 5 divided into three insulating rings 5a, 5b, 5c. The outer surface of each of the electrode bodies 2, 3, 4 forms a corresponding working surface 12, 13, 14. Via a respective electrical supply line 22, 23, 24, each of the electrode bodies 2, 3, 4 and thus each of the corresponding working surfaces 12, 13, 14 may be supplied with high-frequency AC voltage (activated) or be switched to zero potential (deactivated). The electrical supply lines 22, 23, 24 pass through a common head support 6, which is insulating towards the exterior, but they are isolated from each other. This may e.g. be accomplished by way of a multi-core cable. The insulating head support 6 is able perform mechanical and insulating functions. It is, however, also possible for separate elements adopting those functions. For example, a wire may provide for mechanical stability and insulation may be attained using a PTFE tube.

Two of the electrode bodies 2, 3 are configured as rings, such that the electrical supply lines 22, 23, 24 may be conducted from the inside to the electrode body 2, 3, 4. The electrode head 1 may be manufactured by assembling the insulating and electrode rings 5a, 5b, 5c, 2, 3 and the third electrode body 4, which covers the body like a cap.

Due to the separate supply lines, it is possible e.g. to activate exclusively the intermediate working surface 14 for plasma ignition. By additional activation of one of the other working surfaces 12 or 13, the total working surface may be increased, respectively.

In case of the electrode shown in FIGS. 1a, 1b, the quasi-bipolar technique is continued to be used. Instead of a static active electrode, however, several areas (working surfaces 12, 13, 14) are utilized, which may be supplied dynamically with the active potential. Thus, as described above, only the central area 14 may be employed for initial plasma ignition, while the outer areas may be switched on later. To achieve this, automated control via the RF generator (not shown in FIGS. 1a, 1b) may be employed. Depending upon impedance measurements via suitable sensors, known per se from the prior art, the generator may switch individual areas 12, 13, 14 on and off. Thus, e.g. only the areas contacting tissue are ignited. Thus, the input of heat into the saline solution is reduced significantly.

The electrode heads depicted in FIGS. 2 and 3 are of similar construction as shown in FIG. 1a and comprise substantially the same arrangement of the working surfaces 12, 13, 14 as shown in FIG. 1b. There is provided, however, a solid base member as insulating member 5. The variant as shown in FIG. 2 may be manufactured, e.g. by casting the electrode bodies 2, 3, 4 with a high-temperature-resistant plastic, by inserting electrode bodies 2, 3, which are divided inherently, into a ceramic base member (followed by attaching the cap-like electrode body 4) or by casting the metallic electrode bodies 2, 3, 4 into a mold comprising as a core insulating member 5. In the variant depicted in FIG. 3, the working surfaces 12, 13, 14 are applied to the base member 5 electrochemically, by deposit welding or another coating method.

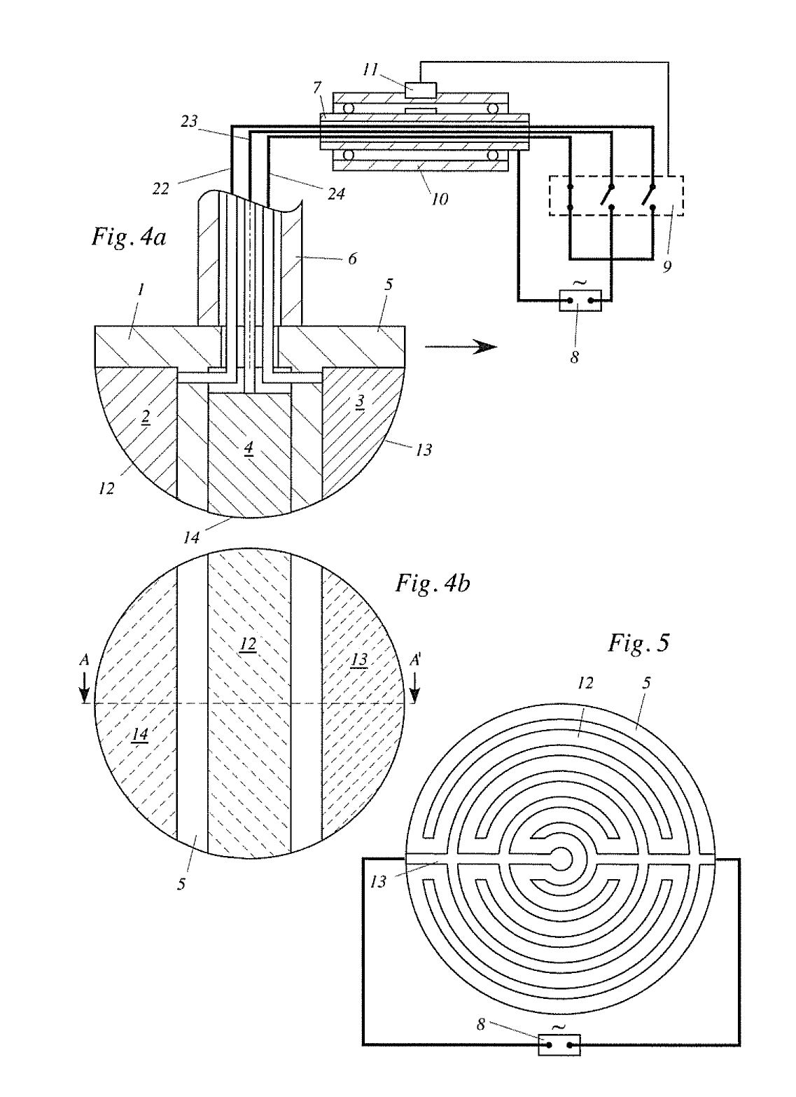

FIG. 4a shows an exemplary embodiment of the electrode head 1 of another surgical vaporization electrode according to the invention. The sectional plane A-A′ is indicated in as a dashed line the plan view in FIG. 1b. The electrode head 1 consists of three metallic electrode bodies 2, 3, 4 inserted into insulator body 5, which in turn is held by a head support 6, which is insulating towards the exterior. The respective electrical supply lines 22, 23, 24 of the electrode bodies 2, 3, 4 are conducted to control- and switching-device 9 via head support 6 and the electrode shaft 7 rigidly connected thereto (connection not shown). The control- and switching-device 9 may separately connect to the RF voltage source or switch to zero potential each of the respective supply lines 22, 23, 24.

The electrode shaft 7 is guided within a transporter 10. Via the capacitive sensor device 11, the control and switching device 9 may detect the movement of the electrode shaft 7 and thus of the electrode head 1 relative to the transporter 10.

In this exemplary embodiment, the working surfaces 12, 13, 14 formed by electrode bodies 2, 3, 4 are situated next to each other or one behind the other, respectively. Thus, working surface 13, leading in the direction of movement, may be activated (indicated as an arrow in FIG. 4a), while trailing working surface 12 may remain at zero potential, such that no thermal energy is introduced into the free saline solution there. The intermediate working surface 14 may either be connected to the respective leading working surface 13 (or, respectively, 12 in the opposite direction of movement), or else remain at zero potential. Obviously, such an electrode may also be implemented with only two working surfaces. Alternatively and differently from what is shown, the intermediate working surface 14 is implemented being considerably smaller than the remaining working surfaces 12, 13 and is used for plasma ignition.

In general, all of the described exemplary embodiments are may be implemented in a similar or modified form having either more or fewer than three working surfaces 12, 13, 14.

The electrode head 1, depicted in FIG. 5 in a plan view from the bottom, comprises two working surfaces 12, 13 in accordance with an actual bipolar electrode. These may be manufactured, e.g. lithographically, exhibiting more complicated outlines. Working surfaces 12, 13 are depicted, which are structured as annulus sector-shaped, concentrically arranged areas, when projected in a plane. A further area is situated centrally, which is disc-shaped in a planar projection. Spatially, the side of the electrode head 1 depicted is either hemispherical or curved, in correspondence to an alternative part of a spherical surface. Therein, plasma is ignited alternately at both poles 12, 13. If the individual concentric zones are situated close enough with respect to each other a continuous plasma layer will result.

1. Surgical vaporization electrode, comprising an electrode head with at least two electrically conductive working surfaces, arranged as to be electrically isolated from each other.

2. A surgical vaporization electrode according to claim 1, wherein each of the working surfaces has at least one substantially annulus-shaped, annulus sector-shaped, elliptical annulus-shaped or elliptical annulus sector-shaped surface portion, when projected in a plane, and the surface portions are arranged concentrically or approximately concentrically in relation to each other, when projected in a plane.

3. Surgical vaporization electrode according to claim 1, wherein the working surfaces are applied in layers to an insulating base member.

4. Surgical vaporization electrode according to claim 1, wherein the electrode head is composed of electrically conductive and electrically non-conductive members.

5. A surgical instrument comprising a surgical vaporization electrode according to claim 1 and an RF generator, wherein the RF generator is configured and connected to the surgical vaporization electrode as to allow for activation and deactivation of the working surfaces separately from each other.

6. The surgical instrument of claim 5, further comprising an electronic control for activating and deactivating the working surfaces.

7. Surgical instrument according to claim 6, further comprising movement detection means for detecting a relative movement of the electrode head with respect to a reference system, wherein the electronic control is adapted to activate and/or deactivate at least one of the working surfaces depending upon the relative movement of the electrode head.

8. Surgical instrument according to claim 7, wherein the electronic control is configured to activate at least one working surface leading with respect to the direction of movement of the electrode head and to deactivate at least one working surface trailing with respect to the direction of movement of the electrode head.

9. The surgical instrument of claim 6, further comprising means for measuring impedance, wherein the electronic control is configured to activate and/or deactivate at least one of the working surfaces depending upon the impedance measurements.

10. A surgical instrument comprising a surgical vaporization electrode according to claim 1 and an RF generator, wherein the RF generator is configured and connected to the surgical vaporization electrode such that two working surfaces operate as alternating poles in bipolar mode.

11. A surgical instrument according to claim 5, wherein the surgical instrument is configured such that for plasma ignition, a predetermined working surface is activated prior to the activation of one or more of the remaining working surfaces.
